(19) United States
(12) Patent Application Publication
(10) Pub. No.: US 2026/0203982 A1
(43) Pub. Date: Jul. 16, 2026

(54) CT SYSTEM AND STORING MEDIUM

(71) Applicant: GE Precision Healthcare LLC, Waukesha, WI (US)

(72) Inventors: Yasuhiro Imai, Hino (JP); Risa Shigemasa, Hino (JP)

(21) Appl. No.: 19/355,831

(22) Filed: Oct. 10, 2025

(30) Foreign Application Priority Data
Oct. 11, 2024 (JP) 2024-179480

(51) Int. Cl. G06T 11/00; A61B 6/00; A61B 6/03

(52) U.S. Cl. CPC G06T 12/30; A61B 6/032; A61B 6/482; A61B 6/5217; G06T 2211/408; G06T 2211/441

(57) ABSTRACT

The CT system includes an X-ray tube, and executes the following: reconstructing a first CT image based on data obtained by scanning a subject under a scanning condition in which a first tube voltage is applied to the X-ray tube, inferring a virtual monochromatic X-ray image of a second energy different from a first energy corresponding to the first tube based on the first CT image, and determining a CT value of a virtual monochromatic X-ray image of a third energy based on a first coefficient defining the relationship between a second CT value corresponding to the second energy and a third CT value corresponding to the third energy, with reference to a first CT value corresponding to the first energy.

### CROSS-REFERENCE TO RELATED APPLICATIONS

This application claims priority to Japanese Application No. 2024-179480, filed on Oct. 11, 2024, the disclosure of which is incorporated herein by reference in its entirety.

### TECHNICAL FIELD

The present invention relates to a CT system for generating a virtual monochromatic X-ray image of an imaging target (subject or the like) and a storing medium storing an instruction for generating a virtual monochromatic X-ray image of an imaging target.

### BACKGROUND ART

A CT device is known as a medical device that noninvasively images a subject. CT devices can acquire tomographic images of a subject in a short scanning time, and thus are widely used in hospitals and other medical facilities.

The CT device applies a prescribed voltage to the cathode-anode tube of an X-ray tube to generate X-rays. The generated X-rays penetrate the subject and are detected by a detector. The CT device reconstructs a CT image of the subject based on the data detected by the detector.

### SUMMARY OF THE INVENTION

Single-energy CT (SECT) is a well-known imaging technology for CT devices. SECT is a method of obtaining CT images of a subject by applying a prescribed voltage (e.g., 120 kVp) to the cathode-anode tube of the X-ray tube to generate X-rays. However, in SECT, CT values may be close even for different materials, and identification of different materials may be difficult.

Therefore, DECT (Dual-Energy CT) technology is being researched and developed. DECT is a technology that uses X-rays in different energy regions to distinguish materials, and DECT-compatible CT devices are commercially available. The DECT technology has a wide range of applications and can be used, for example, to reconstruct virtual monochromatic X-ray images at each energy.

On the other hand, many medical institutions have not yet introduced DECT-compatible CT devices. Therefore, in order to provide diagnosis using a virtual monochromatic X-ray image even to medical institutions that do not have DECT-compatible CT devices, technologies for inferring a virtual monochromatic X-ray image from an image acquired by SECT using deep learning have also been researched and developed.

For example, a technology has been developed for inferring an image at 50 keV based on an image obtained when a tube voltage of 120 kV is applied to an X-ray tube. This technology uses a trained neural network created using AI technology to infer a 50 keV virtual monochromatic X-ray image. This trained neural network is created, for example, as follows.

When creating such a trained neural network, a large amount of training data is prepared using a 120 kV CT image and 50 keV virtual monochromatic X-ray image, and the neural network training is executed using this training data. This training allows for the creation of a trained neural network that infers a 50 keV virtual monochromatic X-ray image from a 120 kV CT image. The trained neural network thus created is able to infer a 50 keV virtual monochromatic X-ray image from a 120 kV CT image obtained by single-energy technology. It is known that a CT image obtained with a tube voltage of 120 kV generally exhibits properties similar to those of a 70 kV virtual monochromatic X-ray image, although this depends on the type of CT device. Therefore, by inferring the 50 keV virtual monochromatic X-ray image using a trained neural network, a user can compare a 120 kV CT image (70 keV virtual monochromatic X-ray image) with the 50 keV virtual monochromatic X-ray. Therefore, improved diagnostic performance can be expected.

Note that the aforementioned technology can only infer 50 KeV images, but if an image at a keV other than 50 keV could also be inferred, further contribution to improving diagnostic performance can be expected. For example, by creating a trained neural network that infers an image at a keV other than 50 keV in addition to a trained neural network that infers a 50 KeV image, an image at a keV other than 50 keV as well as at 50 keV can be inferred, which is expected to further contribute to improving diagnostic performance.

However, the method above requires the creation of a trained neural network for virtual monochromatic X-ray images for each energy. Therefore, training data must be prepared for each virtual monochromatic X-ray image of the energy to be inferred and the neural network must be trained. This poses a problem of an increased number of man-hours for development.

Therefore, a technology is desired, which can generate virtual monochromatic X-ray images of various energy levels (keV) with a low number of man-hours for development.

A first aspect of the present examples include a CT system including an X-ray tube to which a tube voltage is applied, and one or a plurality of processors that executes the following: reconstructing a first CT image based on data obtained by scanning a subject under a scanning condition in which a first tube voltage is applied to the X-ray tube, inferring a virtual monochromatic X-ray image of a second energy different from a first energy corresponding to the first tube voltage based on the first CT image, and determining a CT value of the virtual monochromatic X-ray image of a third energy based on a first coefficient defining the relationship between a second CT value corresponding to the second energy and a third CT value corresponding to the third energy, with reference to a first CT value corresponding to the first energy.

Furthermore, a second aspect of the present examples includes one or more non-transitory computer-readable storing medium storing an instruction, the storing medium causing the one or plurality of processors to, when the instruction is executed by the one or plurality of processors, execute an operation including: reconstructing a first CT image based on data obtained by scanning a subject under a scanning condition in which a first tube voltage is applied to the X-ray tube, inferring a virtual monochromatic X-ray image of a second energy different from a first energy corresponding to the first tube voltage based on the first CT image, and determining a CT value of the virtual monochromatic X-ray image of a third energy based on a first coefficient defining the relationship between a second CT value corresponding to the second energy and a third CT value corresponding to the third energy, with reference to a first CT value corresponding to the first energy.

### Effect of the Invention

In the present invention, a virtual monochromatic X-ray image of an arbitrary energy (e.g., 40 keV) desired by a user can be calculated by using a first coefficient that defines the relationship between a second CT value corresponding to the second energy and a third CT value corresponding to the third energy, with reference to a first CT value corresponding to the first energy. Therefore, it is not necessary to create a trained neural network for each virtual monochromatic X-ray image of each energy. Thus, the number of man-hours for development can be reduced, and development costs can be significantly reduced.

### BRIEF DESCRIPTION OF DRAWINGS

FIG. 1 is a block diagram of a CT system 100 of one examples;

FIG. 2 is a schematic diagram of a curve representing a change in a CT value with respect to energy (keV);

FIG. 3 is an explanatory diagram of a method for creating a curve 12;

FIG. 4 is a diagram depicting the curve 12;

FIG. 5 is a diagram depicting a 50 keV position by a dashed line 22;

FIG. 6 is a diagram depicting a 40 keV position by a dashed line 23;

FIG. 7 is an explanatory diagram of a relationship between CT values;

FIG. 8 is an explanatory diagram showing relationships of the CT value of the curve 11;

FIG. 9 is an explanatory diagram for calculating a CT value of a 45 keV virtual monochromatic X-ray image;

FIG. 10 is an explanatory diagram of a lookup table LUT1 stored in a storing device;

FIG. 11 is a flowchart for acquiring a virtual monochromatic X-ray image of a subject at an arbitrary energy;

FIG. 12 is a diagram schematically depicting a CT image 31 acquired by scanning a subject;

FIG. 13 is a diagram schematically depicting an inferred 50 keV virtual monochromatic X-ray image 32;

FIG. 14 is an explanatory diagram of equations used to create a 40 keV virtual monochromatic X-ray image;

FIG. 15 is an explanatory diagram of a CT value v₄₀ calculated using equation (9);

FIG. 16 is a flowchart of step ST3 in one example;

FIG. 17 is an explanatory diagram of step ST3;

FIG. 18 is an explanatory diagram of a problem that occurs when a subject is scanned at a tube voltage of 100 kV;

FIG. 19 is an explanatory diagram of a principle of generating a 50 keV virtual monochromatic X-ray image from a 100 kV CT image in one example;

FIG. 20 is an explanatory diagram of a lookup table LUT2 stored in a storing device;

FIG. 21 is a flowchart for acquiring a 50 keV virtual monochromatic X-ray image; and

FIG. 22 is an explanatory diagram of the flow of FIG. 21.

### BRIEF DESCRIPTION OF DRAWINGS

Examples for carrying out the invention will be described below, but the present invention is not limited to the following examples.

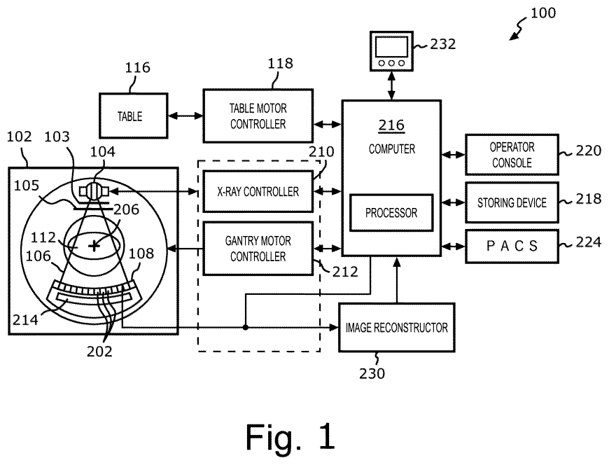

FIG. 1 is a block diagram of a CT system 100 of one example. The CT system 100 has a gantry 102. The gantry 102 has an opening, a subject (imaging target) 112 moves into the opening, and a scan of the subject 112 is executed. The gantry 102 is equipped with an X-ray tube 104, a filter part 103, a pre-collimator 105, an X-ray detector 108, and the like. The X-ray tube 104 generates X-rays when a prescribed voltage is applied to a cathode-anode tube. The filter part 103 includes, for example, a flat plate filter and/or a bow-tie filter. The pre-collimator 105 is a member for narrowing the X-ray irradiation range such that X-rays are not irradiated in unwanted regions.

The X-ray detector 108 includes a plurality of detector elements 202. The plurality of detector elements 202 detect an X-ray beam 106 that is irradiated from the X-ray tube 104 and passes through the subject 112, such as a patient or the like. Therefore, the X-ray detector 108 can acquire projection data for each view.

The projection data detected by the X-ray detector 108 is acquired by the DAS 214. The DAS 214 executes prescribed processing, including sampling, digital conversion, and the like, on the acquired projection data. The processed projection data is transmitted to a computer 216. The computer 216 stores the data from the DAS 214 in a storing device 218. The storing device 218 includes one or more storing medium that stores a program, instruction, and the like to be executed by the processor. The storing medium may be, for example, one or more non-transitory computer-readable storing medium. The storing device 218 can include, for example, a hard disk drive, floppy disk drive, compact disc read/write (CD-R/W) drive, digital versatile disc (DVD) drive, flash drive, and/or solid-state storage drive.

The computer 216 includes one or a plurality of processors. The computer 216 uses the one or plurality of processors to output commands and parameters to the DAS 214, X-ray controller 210, and/or gantry motor controller 212, to control data acquisition and/or processing and other system operations.

An operator console 220 is linked to the computer 216. An operator can enter prescribed operator inputs related to an operation of the CT system 100 into the computer 216 by operating the operator console 220. The computer 216 receives an operator input, including a command and/or scan parameter, via the operator console 220 and controls system operation based on the operator input. The operator console 220 can include a keyboard (not depicted) or touch screen for the operator to specify a command and/or scan parameter.

The X-ray controller 210 controls the X-ray tube 104 based on a control signal from the computer 216. Furthermore, the gantry motor controller 212 controls the gantry motor based on control signals from the computer 216.

FIG. 1 depicts only one operator console 220, but two or more operator consoles may be linked to the computer 216. Furthermore, the CT system 100 may also allow a plurality of remotely located displays, printers, workstations, and/or similar devices to be linked via, for example, a wired and/or wireless network.

In one example, the CT system 100 may include a Picture Archiving and Communication System (PACS) 224, or may be linked to the PACS 224. In a typical implementation, a PACS 224 may be linked to a remote system such as a radiology department information system, hospital information system, and/or internal or external network (not depicted) or the like.

The computer 216 supplies commands to the table motor controller 118 to control the table 116. The table motor controller 118 can control the table 116 based on commands received. In particular, the table motor controller 118 can move the table 116 such that the subject 112 is properly positioned within the opening of the gantry 102.

As mentioned above, the DAS 214 samples and digitally converts the projection data acquired by the detector elements 202. The image reconstructor 230 then reconstructs the image using the sampled and digitally converted data. The image reconstructor 230 includes one or a plurality of processors, which can execute image reconstruction processing. In FIG. 1, the image reconstructor 230 is depicted as a separate component from the computer 216, but the image reconstructor 230 may form a part of the computer 216. Furthermore, the computer 216 may also perform one or a plurality of functions of the image reconstructor 230. Furthermore, the image reconstructor 230 may be positioned away from the CT system 100 and operatively connected to the CT system 100 using a wired or wireless network.

The image reconstructor 230 can store the reconstructed image in the storing device 218. The image reconstructor 230 may also transmit the reconstructed image to the computer 216. The computer 216 can transmit the reconstructed image and/or patient information to a display 232 communicatively linked to the computer 216 and/or image reconstructor 230.

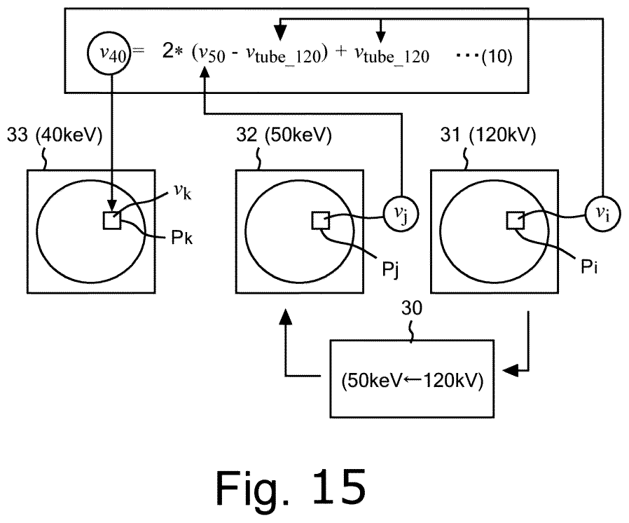

The various methods and processes described in the present specification may be stored as executable instructions on a non-transitory storing medium within the CT system 100 or on an external storing medium communicatively connected to the CT system 100. The executable instructions may be stored on a single storing medium or distributed across a plurality of storing media. One or a plurality of processors provided in the CT system 100 executes the various methods, steps, and processes described in the present specifications in accordance with the instructions stored on a storing medium. For example, in the present example, a processor executes methods, steps, and processes related to a trained neural network 30 described later (the trained neural network 30 is a neural network that infers a 50 keV virtual monochromatic X-ray image from a 120 kV CT image. For example, see FIGS. 13 and 15).

As described above, the CT system 100 can use the trained neural network 30. The trained neural network 30 infers a 50 keV virtual monochromatic X-ray image from a 120 kV CT image. A neural network that infers a 50 keV virtual monochromatic X-ray image from a 120 kV CT image is in and of itself known and is implemented in some CT devices available from GE Healthcare under the name “True Enhance DL”, for example. As described above, the trained neural network 30 can infer a 50 keV virtual monochromatic X-ray image from a 120 kV CT image. Therefore, even with a CT device that is not equipped with dual-energy technology, a 50 keV virtual monochromatic X-ray image can be obtained from a 120 kV CT image obtained by scanning the subject, which is expected to improve diagnostic performance.

Furthermore, the trained neural network 30 can only infer images at 50 keV, but if another trained neural network that can infer images at a keV other than 50 keV could be prepared, further contribution to improving diagnostic performance can be expected. For example, if a trained neural network that infers images at a keV other than 50 keV is created in addition to a trained neural network that infers 50 KeV images, images at keV other than 50 keV as well as images at 50 keV can be inferred, and further contribution to improving diagnostic performance is highly expected.

Therefore, it is conceivable to create a trained neural network for virtual monochromatic X-ray images of each energy and infer virtual monochromatic X-ray images of various energy levels. For example, if a diagnosis is performed with reference images of 40 keV, 50 keV, 60 keV, 70 keV, 80 keV, 90 keV, 100 keV, 110 keV, 120 keV, 130 keV, and 140 keV, a trained neural network can be created for each of the energy levels to infer virtual monochromatic X-ray images for the energy levels. Therefore, virtual monochromatic X-ray images of various energy levels can be inferred, which allows for further improvement in diagnostic performance.

However, the method above requires the creation of a trained neural network for virtual monochromatic X-ray images for each energy. Therefore, training data must be prepared for each virtual monochromatic X-ray image of the energy to be inferred and the neural network must be trained. This poses a problem of an increased number of man-hours for development.

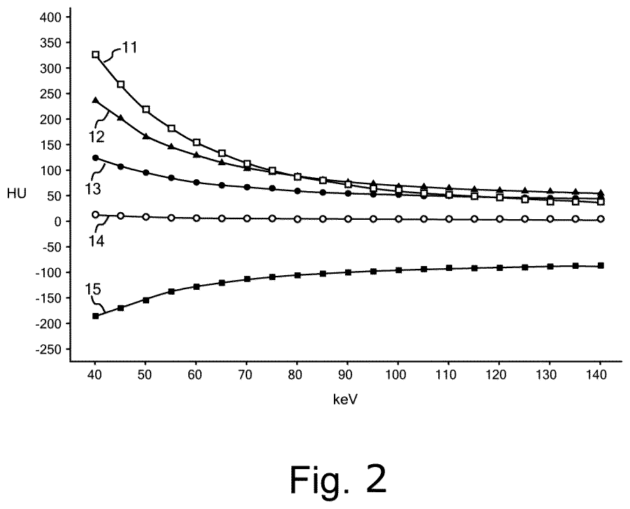

Therefore, the inventors of the present invention have conducted extensive research and have conceived of a method for generating virtual monochromatic X-ray images of various energy levels without creating a trained neural network for each virtual monochromatic X-ray image of each energy. The basic concept of this method will be described below with reference to FIG. 2.

FIG. 2 is a schematic diagram of a curve representing a change in a CT value with respect to energy (keV). FIG. 2 depicts the curves 11 to 15 obtained for each site on a human body. The curve 11 represents a change in the CT value of a kidney with respect to energy (keV). Note that the curve 11 represents a change in the CT value when a contrast agent is flowing into the kidney. The curve 12 represents a change in the CT value of a bone with respect to energy (keV). The curve 13 represents a change in the CT value of a liver with respect to energy (keV). The curve 14 represents a change in the CT value of a cyst of a kidney with respect to energy (keV). The curve 15 represents a change in the CT value of fat with respect to energy (keV).

These curves 11 to 15 can be created based on data obtained by actually scanning a subject using a CT device equipped with dual-energy technology. Note that a phantom containing a common substance included in the human body (e.g., water, iodine, calcium) may be scanned, and CT value curves for each site may be created based on data obtained from the phantom. Furthermore, a CT value curve may be created based on both data obtained by scanning a subject and data obtained by scanning a phantom.

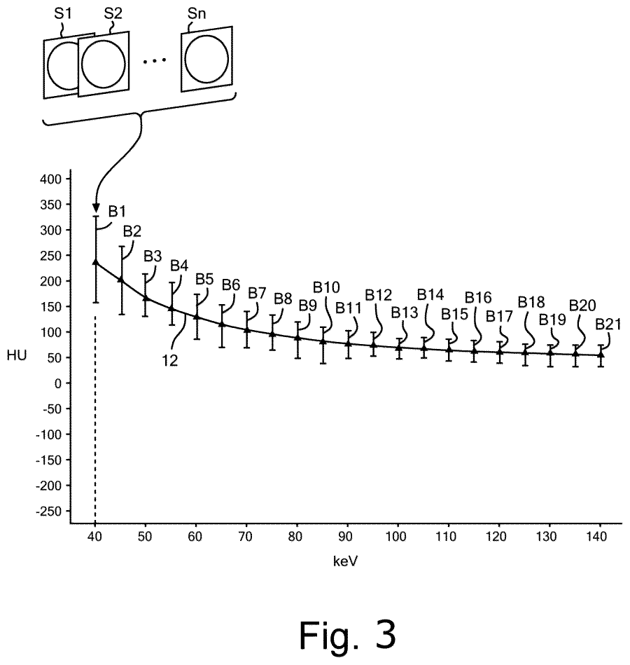

A method for creating the curve 12 will be described below with reference to FIG. 3, taking the curve 12 as a representative example of the curves 11 to 15. The curve 12 is created by first acquiring virtual monochromatic X-ray images in the energy range of 40 keV to 140 keV based on data obtained by scanning a plurality of subjects and/or phantoms using dual-energy technology. In the present example, virtual monochromatic X-ray images are acquired in the range of 40 keV to 140 keV in increments of 5 keV, but virtual monochromatic images of an arbitrary keV can be acquired. For example, a virtual monochromatic X-ray image may be acquired every 1 keV, or every 10 keV. Note that due to space limitations, only the virtual monochromatic X-ray images S1 to Sn obtained by scanning at an energy of 40 keV are depicted herein, but a plurality of virtual monochromatic X-ray images are also acquired for other energy levels. Furthermore, from the virtual monochromatic X-ray image obtained thereby, the CT value of the bone is identified for each energy (keV). For example, with a focus on 40 keV, the CT values of the bone are identified from the virtual monochromatic X-ray images S1 to Sn. In FIG. 3, a bar B1 representing the range of CT value variation is depicted at 40 keV, and the bar B1 represents the CT value distribution range of the plurality of virtual monochromatic X-ray images S1 to Sn. Furthermore, for other energy levels, the CT values are identified in the same manner as for 40 keV. In FIG. 3, bars B2 to B21 representing the range of CT value variation are depicted for 45 keV to 140 keV.

Furthermore, a curve 12 is drawn so as to pass through the range of the bars B1 to B21 depicted for each energy (keV). The curve 12 may be created so as to pass through an intermediate point of the range of CT values defined by each bar, or may be created so as to pass through a point within the range of CT values defined by each bar where the CT values are most densely concentrated.

Note that although a method for creating the curve 12 has been described with reference to FIG. 3, the other curves 11, 13, 14, and 15 can also be created in a similar manner.

Therefore, from the curves 11 to 15, it is possible to know how the CT value changes with respect to the energy (keV). Furthermore, as described above, technologies have been developed to infer a 50 keV virtual monochromatic X-ray image from a 120 kV CT image.

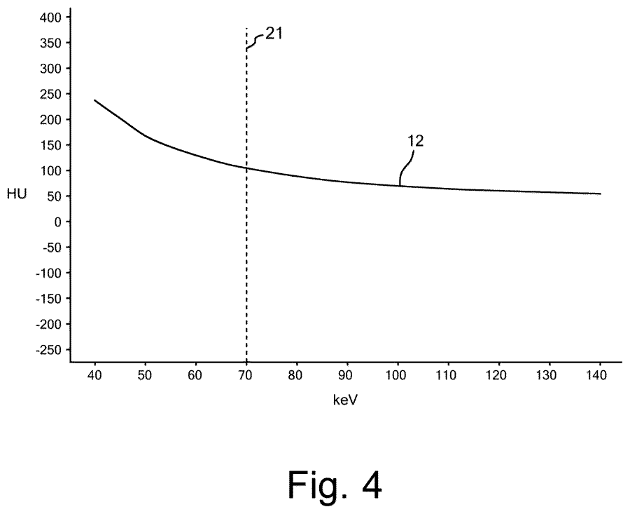

Furthermore, the inventors of the present invention conducted extensive research and discovered that by using the CT values obtained from the curve and a technology for inferring a 50 keV virtual monochromatic X-ray image from a 120 kV CT image, a virtual monochromatic X-ray image of an energy other than 50 keV can be generated from a single-energy CT image without creating a trained neural network for virtual monochromatic X-ray images of each energy (keV). A principle of generating a virtual monochromatic X-ray image of an energy other than 50 keV by using the CT values obtained from the curve and a technology for inferring a 50 keV virtual monochromatic X-ray image from a 120 kV CT image will be specifically described below with reference to FIGS. 4 to 9.

FIG. 4 is a diagram depicting the curve 12. First, the energy (keV) of a virtual monochromatic X-ray image corresponding to the tube voltage used in single-energy imaging is determined. Although various voltage values can be used as the tube voltage for single-energy imaging, a case in which 120 kV is used as the tube voltage for single-energy imaging is considered herein. In general, the characteristics (e.g., contrast and the like) of a 120 kV CT image can be considered sufficiently similar to a 70 keV virtual monochromatic X-ray image. Therefore, the energy of a virtual monochromatic X-ray image corresponding to 120 kV can be assumed to be 70 keV.

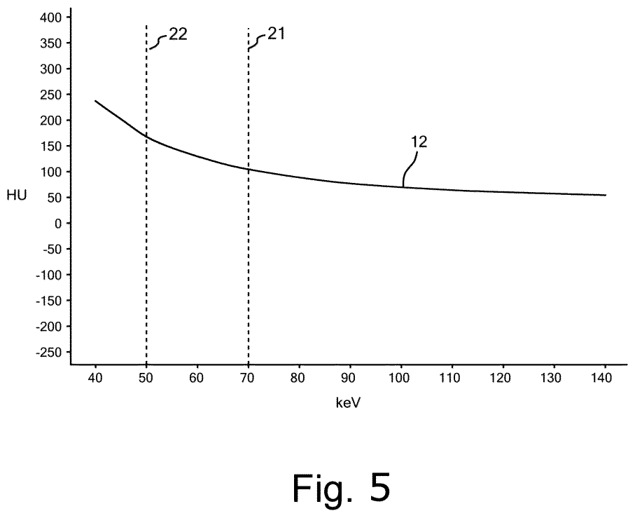

Next, the position representing 70 keV on the horizontal axis (keV axis) of the curve 12 is identified. In FIG. 4, a 70 keV position is indicated by a dashed line 21. Next, the energy (keV) position of the virtual monochromatic X-ray image that the trained neural network infers is identified from the horizontal axis (keV axis) of the curve 12. In the present example, the energy of the virtual monochromatic X-ray image inferred by the trained neural network is 50 keV, and therefore, a position representing 50 keV is identified on the horizontal axis (keV axis) of the curve 12. In FIG. 5, a 50 keV position is indicated by a dashed line 22.

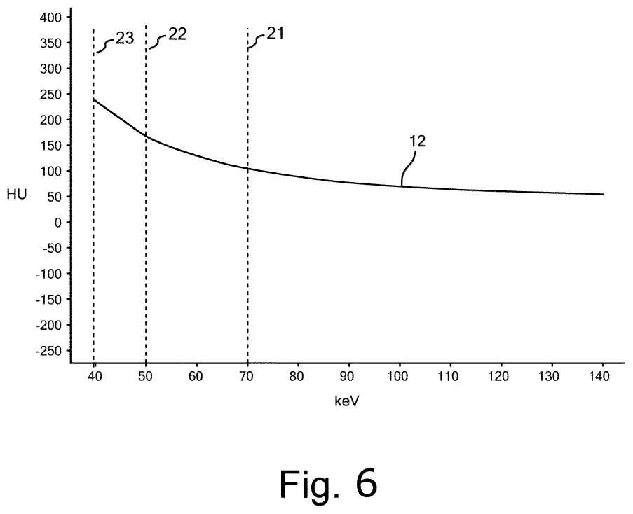

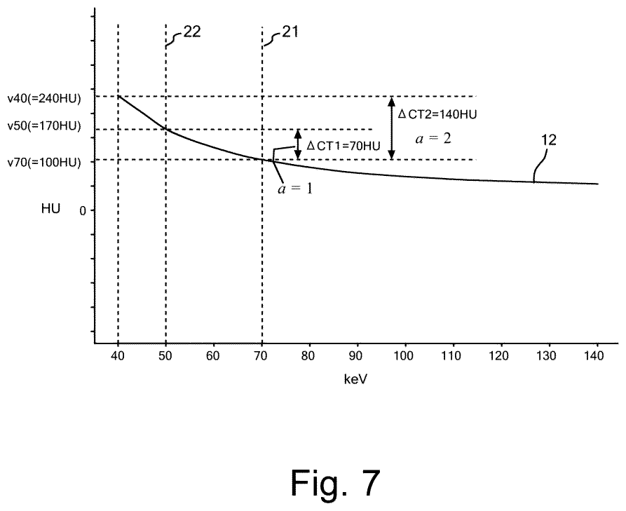

Next, a case in which a virtual monochromatic X-ray image at an energy other than the 50 keV virtual monochromatic X-ray image is generated is considered. Herein, a case in which a 40 keV virtual monochromatic X-ray image energy is generated is considered. Therefore, the position representing 40 keV on the horizontal axis (keV axis) of the curve 12 is identified. In FIG. 6, a 40 keV position is indicated by a dashed line 23. Next, a relationship between CT values at 70 keV, 50 keV, and 40 keV is examined (see FIG. 7).

FIG. 7 is an explanatory diagram of a relationship between CT values. In FIG. 7, the CT value for 70 keV is represented by “v₇₀”, where v₇₀≈100 HU. Furthermore, the CT value for 50 keV is represented by “v₅₀”, where v₅₀≈170 HU. Furthermore, the CT value for 40 keV is represented by “v₄₀”, where v₄₀≈240 HU. Note that these CT values v₇₀, v₅₀, and v₄₀ may vary slightly depending on the method for creating the curve 12. For example, the CT value at 70 keV may deviate from 100 HU. However, such a deviation in the CT value is sufficiently small to be ignored when describing an effect of the present example. Therefore, in the following description, the CT value at 70 keV is 100 HU, the CT value at 50 keV is 170 HU, and the CT value at 40 keV is 240 HU.

First, a difference ΔCT1 between the CT value v₇₀ at 70 keV and the CT value v₅₀ at 50 keV is calculated. ΔCT1 is expressed by the following equation.

$$Δ CT 1 = {v}_{50} - {v}_{70} \qquad ( 1 )$$

The CT value v₇₀=100 HU and the CT value v₅₀=170 HU, and thus ΔCT1 can be calculated by the following equation:

$$Δ CT 1 = {v}_{50} - {v}_{70} = 170 HU - 100 HU = 70 HU$$

Next, a difference ΔCT2 between the CT value v₇₀ at 70 keV and the CT value v₄₀ at 40 keV is calculated. ΔCT2 can be calculated using the following equation:

$$Δ CT 2 = {v}_{40} - {v}_{70} \qquad ( 2 )$$

The CT value v₇₀=100 HU and the CT value v₄₀=240 HU, and thus ΔCT2 can be calculated by the following equation:

$$Δ CT 2 = {v}_{40} - {v}_{70} = 240 HU - 100 HU = 140 HU$$

Therefore, ΔCT1 and ΔCT2 can be considered to have the following relationship:

$$Δ CT 2 = 2 * Δ CT 1 \qquad ( 3 )$$

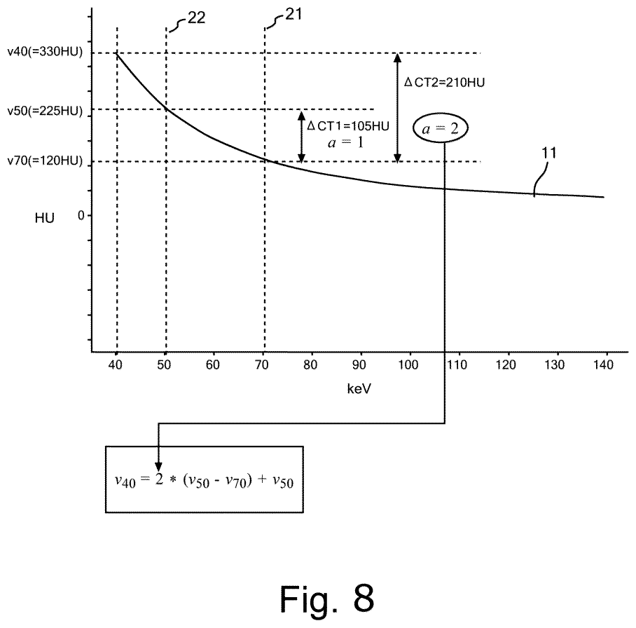

From equation (3), it can be seen that ΔCT2 can be expressed as two times that of ΔCT1. In other words, it can be seen that if the CT value at 70 keV is used as a reference, the CT value at 40 keV is double the CT value at 50 keV. Next, a relationship between CT values at 70 keV, 50 keV, and 40 keV is examined for a curve other than the curve 12 (see FIG. 8).

FIG. 8 is a explanatory diagram of the relationship of the curve 11 with respect to the CT value. The CT value is approximately 120 HU at 70 keV, the CT value is approximately 225 HU at 50 keV, and the CT value is approximately 330 HU at 40 keV.

Next, the difference ΔCT1 between the CT value of 120 HU at 70 keV and the CT value of 225 HU at 50 keV is calculated. ΔCT1 can be calculated using the following equation:

$$Δ CT 1 = 225 HU - 120 HU = 105 HU$$

Furthermore, the difference ΔCT2 between the CT value of 120 HU at 70 keV and the CT value of 330 HU at 40 keV is calculated. ΔCT2 can be calculated using the following equation:

$$Δ CT 2 = 330 HU - 120 HU = 210 HU$$

Therefore, ΔCT1 and ΔCT2 can be considered to have the following relationship:

$$Δ CT 2 = 2 * Δ CT 1$$

Therefore, it can be seen that ΔCT2 for the curve 11 can also be expressed as double ΔCT1. Furthermore, although a detailed description is omitted, ΔCT1 and ΔCT2 for the other CT curves 13 to 15 can also be roughly expressed by the relationship in equation (3). In light of the foregoing, it has been found that ΔCT2 can be calculated by multiplying ΔCT1 by a scaling coefficient of “2”, regardless of the imaging site.

Furthermore, as indicated in equations (1) and (2), ΔCT1 and ΔCT2 are expressed by the following equations:

$$Δ C T 1 = {v}_{50} - {v}_{70}$$

$$Δ CT 2 = {v}_{40} - {v}_{70}$$

Therefore, when equations (1) and (2) are substituted into equation (3), the following equation is obtained:

$$Δ CT 2 = 2 * Δ CT 1$$

$${v}_{40} - {v}_{70} = 2 ( {v}_{50} - {v}_{70} )$$

$${v}_{40} = 2 ( {v}_{50} - {v}_{70} ) + {v}_{70} \qquad ( 4 )$$

Therefore, it can be seen that the CT value of the 40 keV virtual monochromatic X-ray image can be calculated by substituting the CT value v₇₀ of the 70 keV virtual monochromatic X-ray image and the CT value v₅₀ of the 50 keV virtual monochromatic X-ray image into equation (4).

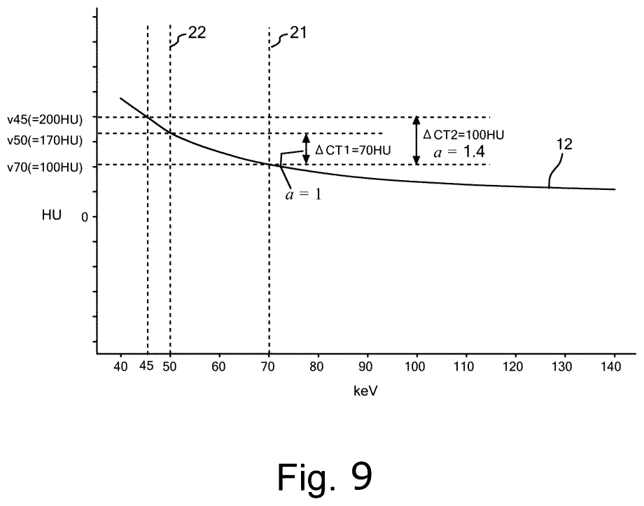

Note that although equation (4) describes an example of calculating the CT value v₄₀ of a 40 keV virtual monochromatic X-ray image, CT values of virtual monochromatic X-ray images of other energy levels can also be calculated in accordance with the description above. For example, a 45 keV virtual monochromatic X-ray image is considered as a virtual monochromatic X-ray image with an energy other than 40 keV, and a case is described in which the CT value of the 45 keV virtual monochromatic X-ray image is calculated. FIG. 9 is an explanatory diagram for calculating the CT value of the 45 keV virtual monochromatic X-ray image.

As described above, the CT value v₇₀ at 70 keV is v₇₀=100 HU, and the CT value v₅₀ at 50 keV is v₅₀=170 HU. Furthermore, the CT value v₄₅ at 45 keV is v₄₅=200 HU. The difference ΔCT1 between the CT value v₇₀ (=100 HU) at 70 keV and the CT value v₅₀ (=170 HU) at 50 keV can be calculated by the following equation:

$$\begin{matrix}Δ CT 1 & = 170 HU - 100 HU \\ & = 70 HU\end{matrix}$$

Furthermore, the difference ΔCT2 between the CT value v₇₀ (=100 HU) at 70 keV and the CT value v₄₅ (=200 HU) at 45 keV can be calculated by the following equation:

$$\begin{matrix}Δ CT 2 & = 200 HU - 100 HU \\ & = 100 HU\end{matrix}$$

Therefore, ΔCT1 and ΔCT2 can be considered to have the following relationship:

$$Δ CT 2 \approx 14 * Δ CT 1 \qquad ( 5 )$$

From equation (5), it can be seen that for the 45 keV virtual monochromatic X-ray image, ΔCT2 can be calculated by multiplying ΔCT1 by a scaling coefficient of 1.4. Therefore, the CT value v₄₅ of the 45 keV virtual monochromatic X-ray image can be calculated by the following equation:

$$v 45 = 1.4 ( {v}_{50} - {v}_{70} ) + {v}_{70} \qquad ( 6 )$$

Therefore, by comparing equations (4) and (6), it can be seen that the CT value v₄₅ of the 45 keV virtual monochromatic X-ray image can be calculated simply by changing the scaling coefficient in equation (4) from “2” to “1.4”.

Therefore, in equations (4) and (6), if the CT values v₄₀ and v₄₅ are replaced with “v_{E}” representing the CT value of a virtual monochromatic X-ray image of arbitrary energy E, and further, the scaling coefficients “2” and “1.4” are replaced with a scaling coefficient “a”, then equations (4) and (6) can be generalized to the following equations:

$${v}_{E} = a ( {v}_{50} - {v}_{70} ) + {v}_{70} \qquad ( 7 )$$

In equation (7), v₇₀ is the CT value of the 70 keV virtual monochromatic X-ray image, and v₅₀ is the CT value of the 50 keV virtual monochromatic X-ray image. Furthermore, a is a scaling coefficient, and the scaling coefficient a defines a relationship between a CT value corresponding to 40 keV and a CT value corresponding to 50 keV, with a CT value corresponding to 70 keV as a reference. The scaling coefficient a is a value determined depending on the value of the energy E, and therefore, it can be seen that CT value v_{E} of the virtual monochromatic X-ray image of arbitrary energy E can be calculated simply by changing the value of the scaling coefficient a.

Note that as described above, the 70 keV virtual monochromatic X-ray image corresponds to the 120 kV CT image. Therefore, when the CT value of the 120 kV CT image is represented by the symbol “v_{tube_120}”, v₇₀ in equation (7) can be replaced with v_{tube_120}. In other words, equation (7) can be expressed as follows:

$${v}_{E} = a ( {v}_{50} - {v}_{tube \_ 120} ) + {v}_{tube \_ 120} \qquad ( 8 )$$

Where, v_{tube_120}: CT value of 120 kV CT image
v₅₀: CT value of 50 keV virtual monochromatic X-ray image
v_{E}: CT value of virtual monochromatic X-ray image of energy E

Therefore, it can be seen that if the CT value v_{tube_120} of the 120 kV CT image and the CT value v₅₀ of the 50 keV virtual monochromatic X-ray image are known, the CT value v_{E} of the virtual monochromatic X-ray image of energy E can be calculated.

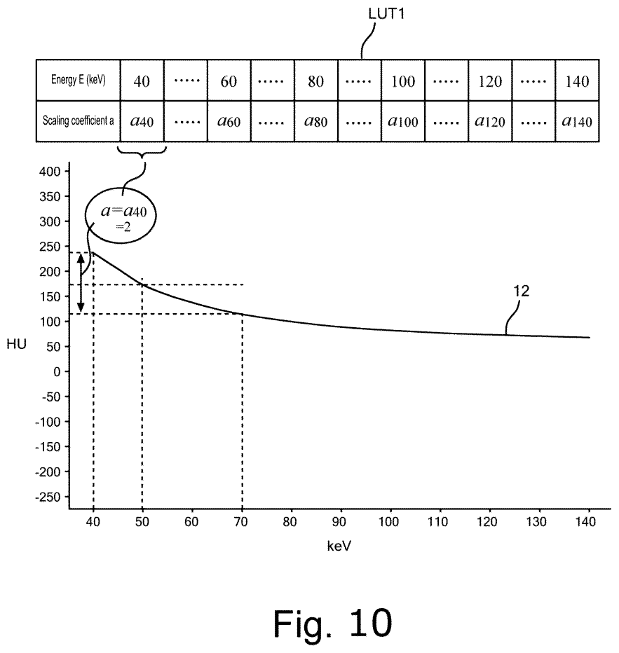

In the present example, the storing device stores a lookup table indicating a correspondence relationship between the energy E of a virtual monochromatic X-ray image and the scaling coefficient a (see FIG. 10).

FIG. 10 is an explanatory diagram of a lookup table LUT1 stored in a storing device. The lookup table LUT1 has a column for the energy E (keV) of the virtual monochromatic X-ray image and a column for the scaling coefficient a. In FIG. 10, for convenience of description, 40 keV, 60 keV, 80 keV, 100 keV, 120 keV, and 140 keV are depicted as examples of the energy E (keV), but the energy is not limited to these energy levels, and an arbitrary energy within the range of 40 keV to 140 keV can be used as an example of the energy E (keV) in the lookup table LUT1. Furthermore, an energy less than 40 keV and/or greater than 140 keV may also be used as an example of the energy E (keV) in the lookup table LUT1.

Furthermore, in the scaling coefficient a column, the scaling coefficient a corresponding to each energy E is indicated as “a₄₀”, “a₆₀”, “a₈₀”, “a₁₀₀”, “a₁₂₀”, and “a₁₄₀”, respectively. For example, the scaling coefficient a₄₀ for energy E=40 keV is a₄₀=2 as described above. Note that although specific values of the other scaling coefficients “a₆₀”, “a₈₀”, “a₁₀₀”, “a₁₂₀”, and “a₁₄₀” are not indicated herein, the values of the other scaling coefficients can also be determined in the manner described with reference to FIGS. 7 to 9.

Therefore, the lookup table LUT1 represents a correspondence relationship between the virtual monochromatic X-ray image energy levels 40 keV to 140 keV and the scaling coefficients a₄₀ to a₁₄₀. In the present example, equation (8) and the lookup table LUT1 are used to generate a virtual monochromatic X-ray image of a subject at an arbitrary energy. The flow of generating a virtual monochromatic X-ray image of a subject at an arbitrary energy in the present example will be described below.

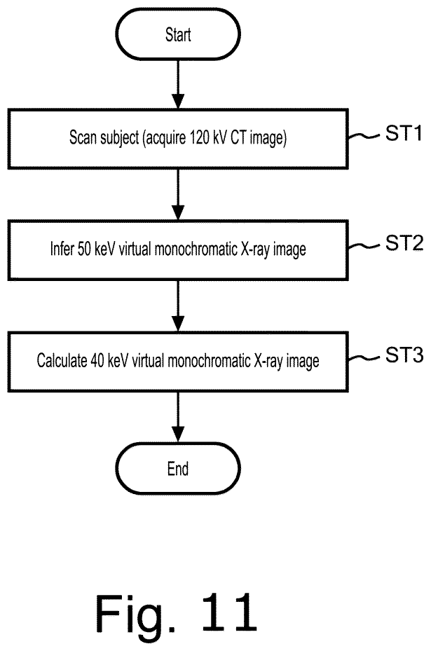

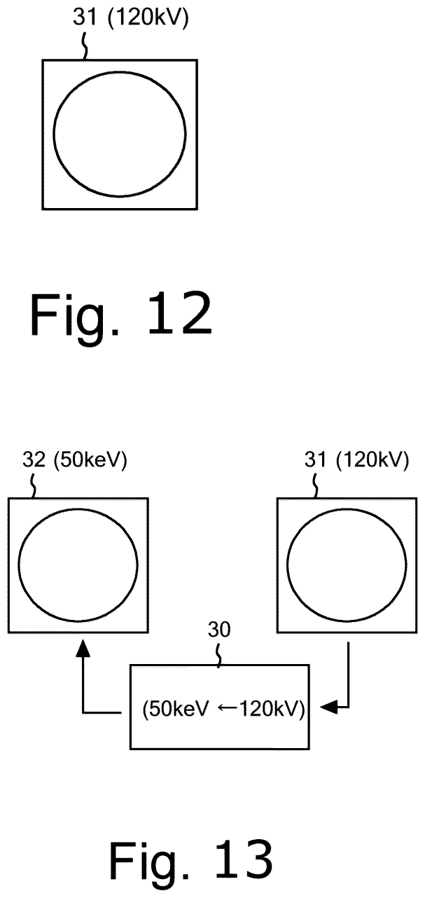

FIG. 11 is a flowchart for acquiring a virtual monochromatic X-ray image of an arbitrary energy, and FIGS. 12 to 15 are explanatory diagrams of each step executed in the flow of FIG. 11.

In step ST1, a subject is scanned under a scanning condition in which a tube voltage of 120 kV is applied to the X-ray tube. The processor reconstructs a CT image of the subject based on data obtained by scanning the subject. FIG. 12 schematically depicts a CT image 31 acquired by scanning the subject. After scanning the subject, the process proceeds to step ST2.

In step ST2, the processor uses the trained neural network 30 to infer a 50 keV virtual monochromatic X-ray image from the CT image 31. FIG. 13 schematically depicts an inferred 50 keV virtual monochromatic X-ray image 32. The processor can input the CT image 31 into the trained neural network 30 to infer the 50 keV virtual monochromatic X-ray image 32. Furthermore, the processor may execute pre-processing on the CT image 31, if necessary, and input the pre-processed CT image 31 into the trained neural network 30 to infer the 50 keV virtual monochromatic X-ray image 32. After the 50 keV virtual monochromatic X-ray image 32 is inferred, the process proceeds to step ST3.

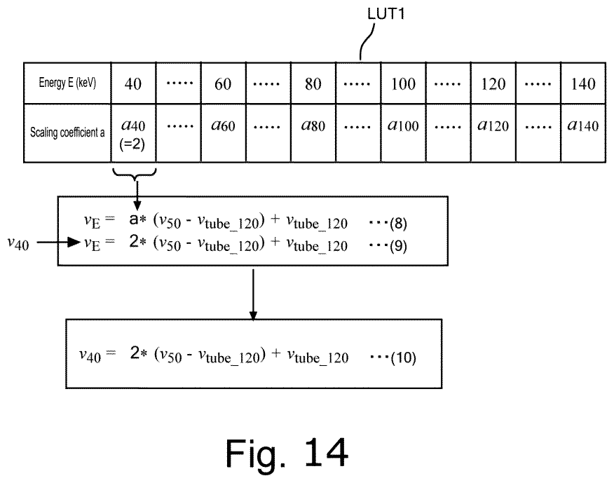

In step ST3, the processor generates a virtual monochromatic X-ray image of another energy different from the 50 keV virtual monochromatic X-ray image 32 based on the CT image 31 and the 50 keV virtual monochromatic X-ray image 32. Step ST3 will be described with reference to FIGS. 14 and 15.

FIG. 14 is an explanatory diagram of equations used to generate virtual monochromatic X-ray images of other energy levels than the 50 keV virtual monochromatic X-ray image 32. Note that hereinafter, 40 keV is considered as an example of another energy, but the same description can be applied to energy levels other than 40 keV.

First, the processor selects a scaling coefficient a corresponding to 40 keV from among a plurality of scaling coefficients a₄₀ to a₁₄₀ in the lookup table LUT1 stored in the storing device. The scaling coefficient a corresponding to 40 keV is a=a₄₀. Therefore, the processor selects the scaling coefficient a=a₄₀ from the lookup table LUT1. After selecting the scaling coefficient a₄₀, the selected scaling coefficient a₄₀ is substituted for a in equation (8). Note that a₄₀=2, and thus a=a₄₀=2 is substituted for a in equation (8). Therefore, the following equation is obtained:

$$\begin{matrix}{v}_{E} & = a ( {v}_{50} - {v}_{tube \_ 120} ) + {v}_{tube \_ 120} \\ & = 2 ( {v}_{50} - {v}_{tube \_ 120} ) + {v}_{tube \_ 120}\end{matrix} \qquad ( 9 )$$

Furthermore, a 40 keV virtual monochromatic X-ray image is considered to be generated, and thus v_{E} in equation (9) can be replaced with v₄₀. Therefore, equation (9) becomes the following equation:

$${v}_{40} = 2 ( {v}_{50} - {v}_{tube \_ 120} ) + {v}_{tube \_ 120} \qquad ( 10 )$$

Therefore, the processor can calculate the CT value v₄₀ of the 40 keV virtual monochromatic X-ray image using equation (10). Specifically, the CT value v₄₀ is calculated as follows.

FIG. 15 is an explanatory diagram of the CT value v₄₀ calculated using equation (10). The processor identifies a pixel Pᵢ of interest from the CT image 31. Furthermore, CT value vᵢ of the pixel Pᵢ of interest is read. The CT image 31 is an image obtained by the tube voltage of 120 kV, and therefore, the processor substitutes vᵢ for v_{tube_120} in equation (10).

The processor also reads CT value vⱼ of pixel Pⱼ located at the same position as pixel Pᵢ of the CT image 31 from the 50 keV virtual monochromatic X-ray image 32. The CT value vⱼ of the 50 keV virtual monochromatic X-ray image 32 is a value representing the CT value v₅₀ in equation (10), and thus the processor substitutes vⱼ for v₅₀ in equation (10).

Therefore, the processor can calculate CT value vₖ (=v₄₀) of pixel PK in a 40 keV virtual monochromatic X-ray image 33 that is located at the same position as pixel Pᵢ in the CT image 31.

For example, if vᵢ=95 HU and vⱼ=160 HU, vₖ can be calculated as 225 HU.

In the description above, a procedure for calculating the CT value vₖ of the pixel Pk of the 40 keV virtual monochromatic X-ray image 33 has been described, but the same method can also be used to calculate the CT value vₖ of another pixel of the 40 keV virtual monochromatic X-ray image 33. Therefore, the 40 keV virtual monochromatic X-ray image 33 can be calculated based on the CT image 31 and the 50 keV virtual monochromatic X-ray image 33.

Note that in the description above, an example has been described in which the 40 keV virtual monochromatic X-ray image 33 is calculated as a virtual monochromatic X-ray image with energy different from that of a 50 keV virtual monochromatic X-ray image. However, using equation (8) and the lookup table LUT1, a virtual monochromatic X-ray image of another energy can be calculated in addition to the 40 keV virtual monochromatic X-ray image 33. For example, if desired, a 60 keV virtual monochromatic X-ray image can be calculated based on the following equation:

$${v}_{60} = {a}_{60} ( {v}_{50} - {v}_{tube \_ 120} ) + {v}_{tube \_ 120} \qquad ( 11 )$$

The abovementioned equation (11) is obtained by replacing v_{E} in equation (8) with v₆₀ and by replacing the scaling coefficient a in equation (8) with a scaling coefficient a₆₀ corresponding to an energy of 60 keV. The processor can calculate the CT value of the 60 keV virtual monochromatic X-ray image by substituting the CT value of the CT image 31 for vtube_120 in equation (11) and substituting the CT value of the 50 keV virtual monochromatic X-ray image 32 for v₅₀ in equation (11).

Similarly, virtual monochromatic X-ray images of other energy levels can be calculated. A display device can display not only the 50 keV virtual monochromatic X-ray image 32, but also a virtual monochromatic X-ray image of an arbitrary energy other than 50 keV.

Thus, the user can visually confirm virtual monochromatic X-ray images of various energy levels. In this manner, the flow depicted in FIG. 11 ends.

In one example, by using the lookup table LUT1 (see FIG. 14) expressing a correspondence relationship between the energy E (keV) and the scaling coefficient a, a virtual monochromatic X-ray image of an arbitrary energy (e.g., 40 keV) desired by the user can be calculated. Therefore, in one example, by using the lookup table LUT1, a virtual monochromatic X-ray image of an arbitrary energy desired by the user can be calculated, without creating a trained neural network 30 for a virtual monochromatic X-ray image for each energy in the range of 40 keV to 140 keV. Therefore, a virtual monochromatic X-ray image of an arbitrary energy can be generated without preparing training data for the neural network for each energy in the range of 40 keV to 140 keV or training the neural network for each energy. Therefore, the method of one example using the lookup table LUT1 can reduce the number of man-hours for development and significantly reduce development costs compared to a method of creating a trained neural network 30 for each energy in the range of 40 keV to 140 keV.

Note that in the present example, the scaling coefficient a is selected from the lookup table LUT1 stored in the storing device. However, a CT value data group (e.g., any one of the curves 11 to 15) representing the change in CT value with respect to the energy (keV) of a virtual monochromatic X-ray image may be stored instead of storing the lookup table LUT1, and the scaling coefficient a may be calculated based on the stored CT value data group.

Note that in the present example, the trained neural network 30 infers the 50 keV virtual monochromatic X-ray image 32 from the CT image 31. However, the virtual monochromatic X-ray image to be inferred does not necessarily have to be limited to a 50 keV virtual monochromatic X-ray image, and a virtual monochromatic X-ray image of another energy may be inferred instead of the 50 keV virtual monochromatic X-ray image. If the energy of the virtual monochromatic X-ray image to be inferred is extended to an arbitrary energy other than 50 keV, equation (8) can be generalized to the following equation:

$${v}_{E} = a ( {v}_{E \_ inf} - {v}_{tube \_ 120} ) + {v}_{tube \_ 120} \qquad ( 12 )$$

Herein, v_{E} inf in equation (12) is the arbitrary energy of the virtual monochromatic X-ray image to be inferred.

Therefore, the virtual monochromatic X-ray image to be inferred is not limited to a virtual monochromatic X-ray image of 50 keV, and even if a virtual monochromatic X-ray image of another energy other than 50 keV is inferred, the CT value v_{E} of the virtual monochromatic X-ray image of arbitrary energy E can be calculated. For example, if a trained neural network that infers a 60 keV virtual monochromatic X-ray image from the 120 kV CT image is prepared instead of the trained neural network 30 that infers the 50 keV virtual monochromatic X-ray image from the 120 kV CT image, the CT value v_{E} of a virtual monochromatic X-ray image of an arbitrary energy E desired by the user can be calculated using the 120 kV CT image and the inferred 60 keV virtual monochromatic X-ray image. In this case, equation (12) can be expressed as follows:

$$\begin{matrix}{v}_{E} & = a ( {v}_{E \_ inf} - {v}_{tube \_ 120} ) + {v}_{tube \_ 120} \\ & = a ( {v}_{60} - {v}_{tube \_ 120} ) + {v}_{tube \_ 120}\end{matrix}$$

Herein, v₆₀ represents the CT value of the inferred 60 keV virtual monochromatic X-ray image. Thus, the virtual monochromatic X-ray image to be inferred is not limited to the 50 keV virtual monochromatic X-ray image, but a virtual monochromatic X-ray image of an arbitrary energy other than 50 keV may be inferred.

Note that in the present example, a case where the tube voltage of the X-ray tube is 120 kV is described. However, the tube voltage of the X-ray tube is not limited to 120 kV, and another tube voltage may be used instead of 120 kV. If the tube voltage of the X-ray tube is expanded to an arbitrary tube voltage other than 120 kV, equation (12) can be generalized to the following equation:

$${v}_{E} = a ( {v}_{E \_ inf} - {v}_{tube \_ x} ) + {v}_{tube \_ x} \qquad ( 13 )$$

Herein, v_{tube_x} represents the CT value of the CT image obtained when the tube voltage is x (kV). Therefore, the tube voltage of the X-ray tube is not limited to 120 kV, and even if the tube voltage is other than 120 kV, the CT value of a virtual monochromatic X-ray image of an arbitrary energy desired by the user can be calculated. Thus, the tube voltage is not limited to 120 kV, and a tube voltage other than 120 kV can also be used.

Another example will be described in accordance with the flow depicted in FIG. 11, similarly to the other example. Note that steps ST1 and ST2 in this example are the same as steps ST1 and ST2 in the previous example. Therefore, steps ST1 and ST2 will be described briefly, and step ST3 will be described in detail.

First, steps ST1 and ST2 are executed to acquire the CT image 31 and the 50 keV virtual monochromatic X-ray image 32. The CT image 31 and the 50 keV virtual monochromatic X-ray image 32 acquired in steps ST1 and ST2 are depicted in FIG. 13. After acquiring the CT image 31 and the 50 keV virtual monochromatic X-ray image 32, the process proceeds to step ST3.

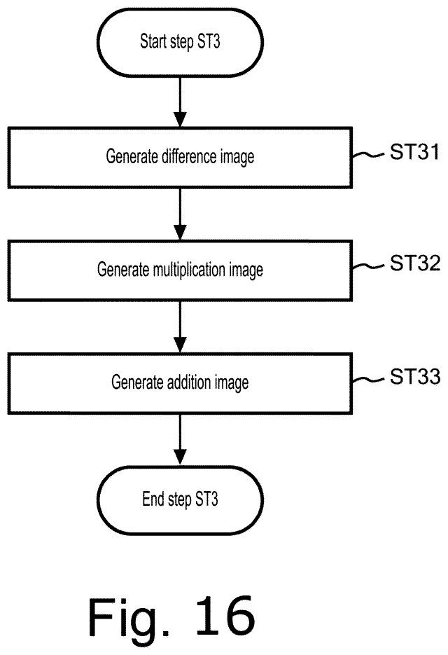

In step ST3, a 40 keV virtual monochromatic X-ray image is generated based on the CT image 31 and the 50 keV virtual monochromatic X-ray image 32. Note that step ST3 in this example is different from step ST3 in a previous example, and thus step ST3 in this example will be described below (see FIG. 16).

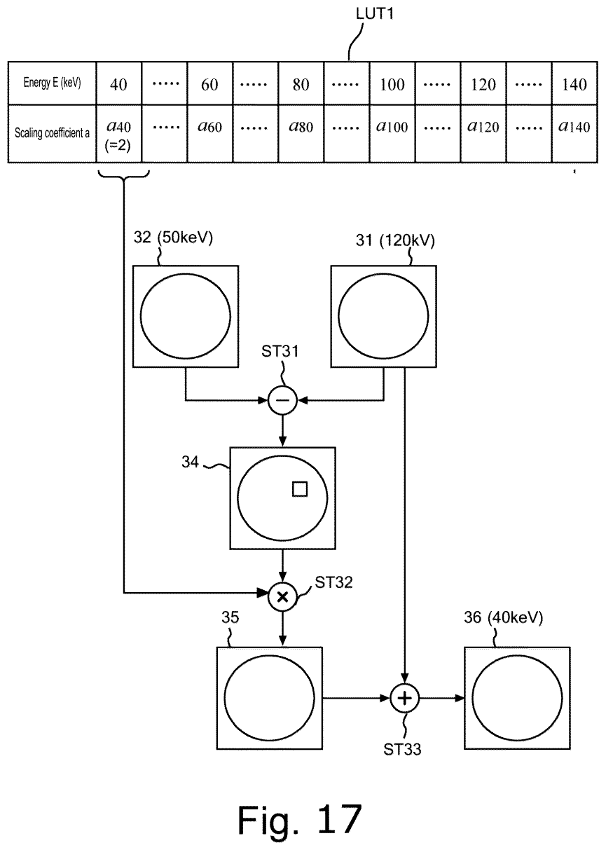

FIG. 16 is a flowchart of step ST3 in this example, and FIG. 17 is an explanatory diagram of step ST3. In step ST31, the processor generates a difference image 34 between the CT image 31 and the virtual monochromatic X-ray image 32. After generating the difference image 34, the process proceeds to step ST32. In step ST32, the processor reads scaling coefficient a₄₀ (=2) corresponding to the energy of 40 keV from the lookup table LUT1. Furthermore, the CT value of each pixel in the difference image 34 is multiplied by the scaling coefficient a₄₀=2 to create a multiplication image 35. After the multiplication image 35 is created, the process proceeds to step ST33.

In step ST33, the processor adds the multiplication image 35 to the CT image 31. Thereby, a 40 keV virtual monochromatic X-ray image 36 can be created. In this example, as in the previous example, by using the lookup table LUT1, a virtual monochromatic X-ray image of an arbitrary energy desired by the user can be calculated. Therefore, in this example as well, there is no need to create a trained neural network 30 for virtual monochromatic X-ray images for each energy (keV), which reduces the number of man-hours for development and significantly reduces development costs.

In the previous examples, examples have been described in which the tube voltage of the X-ray tube is set to 120 kV and a subject is scanned. In yet another example, an example will be described in which the tube voltage of the X-ray tube is set to a different tube voltage from 120 kV and a subject is scanned. Hereinafter, 100 kV is taken as an example of a tube voltage different from 120 kV, and an example of scanning a subject at a tube voltage of 100 kV is described, but the tube voltage may be a tube voltage other than 100 kV.

Note that before specifically describing this example below, a problem that occurs when a subject is scanned at a tube voltage (100 kV) different from 120 kV will be pointed out. After clarifying this problem, this example will be described in detail.

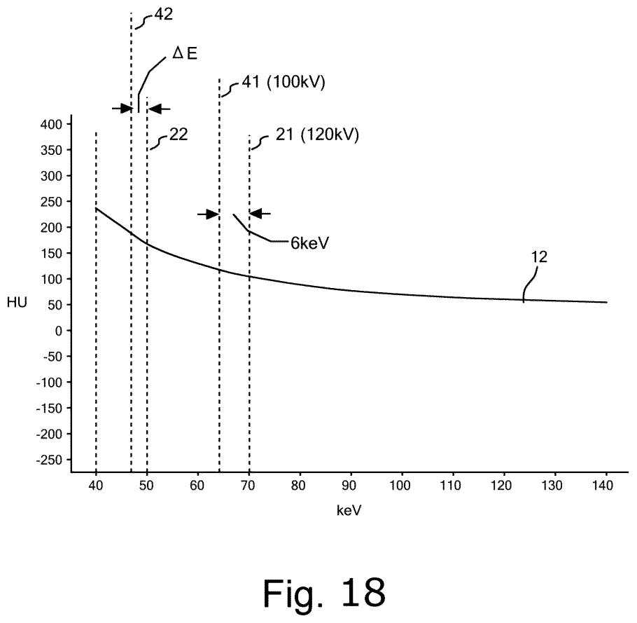

FIG. 18 is an explanatory diagram of the problem that occurs when a subject is scanned at a tube voltage of 100 kV. The 120 kV CT image described in the previous examples is an image corresponding to a 70 keV virtual monochromatic X-ray image (see dashed line 21). On the other hand, a CT image obtained with a tube voltage of 100 kV can generally be considered to be an image corresponding to a 64 keV virtual monochromatic X-ray image. In FIG. 18, a 64 keV position is indicated by a dashed line 41. In other words, when comparing 100 kV and 120 kV, the energy (keV) is not the same, and there is a difference of about 6 keV. Therefore, when a CT image (64 keV) obtained with a tube voltage of 100 kV is input to the trained neural network 30, the virtual monochromatic X-ray image to be inferred is a virtual monochromatic X-ray image of another energy offset from 50 keV by a certain energy ΔE. In FIG. 18, the energy of a virtual monochromatic X-ray image output by inputting the CT image (64 keV) obtained with a tube voltage of 100 kV into the trained neural network 30 is indicated by a dashed line 42. In other words, the energy of the virtual monochromatic X-ray image to be inferred deviates from 50 keV by ΔE, and a problem occurs in which a 50 keV virtual monochromatic X-ray image cannot be obtained.

One method to address this problem is to create a new trained neural network 30 that infers a 50 keV virtual monochromatic X-ray image from a 100 kV CT image, separate from the trained neural network 30 that infers the 50 keV virtual monochromatic X-ray image from the 120 kV CT image. However, this method has the problem that not only the trained neural network 30 for 120 kV but also the trained neural network 30 for 100 kV must be prepared, which increases the number of man-hours for development. Therefore, in this example, the 50 keV virtual monochromatic X-ray image is generated from the 100 kV CT image without creating a trained neural network 30 for 100 kV. This method will be described below.

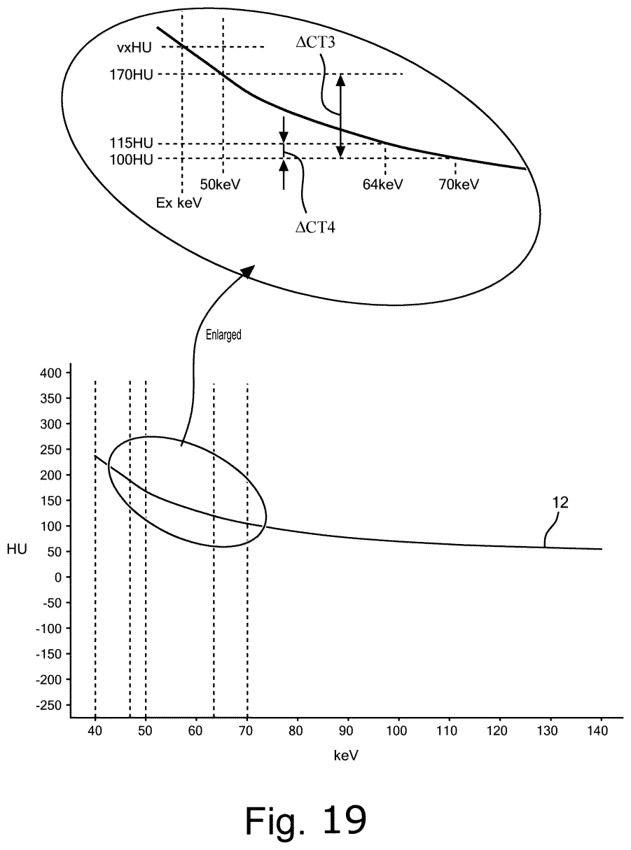

FIG. 19 is an explanatory diagram of a principle of generating a 50 keV virtual monochromatic X-ray image from a 100 kV CT image in this example. First, a difference ΔCT3 between a CT value at 50 keV and a CT value at 70 keV is obtained. The CT value at 50 keV is 170 HU and the CT value at 70 keV is 100 HU, and thus ΔCT3 can be calculated using the following equation:

$$\begin{matrix}Δ CT 3 & = 170 HU - 100 HU \\ & = 70 HU\end{matrix}$$

Next, a difference ΔCT4 between a CT value at 64 keV and a CT value at 70 keV is obtained. The CT value at 64 keV is 115 HU and the CT value at 70 keV is 100 HU, and thus ΔCT4 can be calculated using the following equation:

$$\begin{matrix}Δ CT 4 & = 115 HU - 100 HU \\ & = 15 HU\end{matrix}$$

Therefore, ΔCT3 and ΔCT4 can be considered to have the following relationship:

$$Δ CT 4 / Δ CT 3 = 15 / 70$$

Thus, the CT value at 64 keV is shifted by ΔCT4/ΔCT3=15/70 relative to the CT value at 70 keV. Therefore, by shifting CT value vx of the virtual monochromatic X-ray image of the inferred energy Ex by vx (15/70), the value can be made to match the CT value of a 50 keV virtual monochromatic X-ray image (or can be made to approach the CT value of a 50 kev virtual monochromatic X-ray image).

Therefore, ΔCT4/ΔCT3 can be used as a conversion coefficient b for converting the CT value vx of the virtual monochromatic X-ray image of the inferred energy Ex into the CT value of a 50 keV virtual monochromatic X-ray image. In the example above, the conversion coefficient b is b=15/70, but if necessary, value (15/70) w obtained by multiplying 15/70 by a weighting coefficient w can also be used as the conversion coefficient b. Note that in the description above, the conversion coefficient b is described using the curve 12, but the conversion coefficient b of approximately the same value can be obtained even with the curves 11 and 13 to 15 of other sites. Therefore, regardless of the imaging site, by using the abovementioned conversion coefficient b, the CT value vx of the inferred virtual monochromatic X-ray image can be converted into the CT value of a 50 keV virtual monochromatic X-ray image.

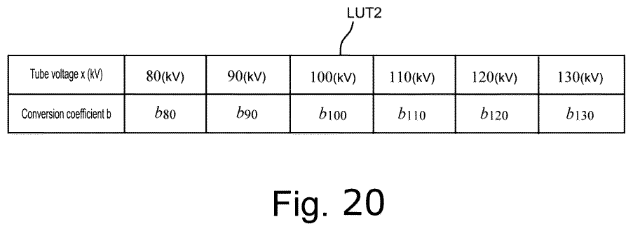

Furthermore, in the description above, a CT image with a tube voltage of 100 kV has been described, but corresponding conversion coefficients b can also be found for other tube voltages in accordance with the description above. In this example, a lookup table expressing a correspondence relationship between a tube voltage x of the X-ray tube and the conversion coefficient b is stored in the storing device (see FIG. 20).

FIG. 20 is an explanatory diagram of a lookup table LUT2 stored in the storing device. The lookup table LUT2 has a column for the tube voltage x of the X-ray tube and a column for the conversion coefficient b. In FIG. 20, 80 kV, 90 kV, 100 kV, 110 kV, 120 kV, and 130 kV are indicated as examples of the tube voltage x, but the energy is not limited thereto, and an arbitrary tube voltage can be used as an example of the tube voltage x in the lookup table LUT2.

Furthermore, in the conversion coefficient b column, the conversion coefficient b corresponding to each tube voltage is indicated as “b₈₀”, “b₉₀”, “b₁₀₀”, “b₁₁₀”, “b₁₂₀”, and “b₁₃₀”, respectively. Therefore, the lookup table LUT2 represents a correspondence relationship between tube voltages x=80 kV to 130 kV and conversion coefficients b=b₈₀ to b₁₃₀. A flow for acquiring the 50 keV virtual monochromatic X-ray image using the lookup table LUT2 in this example will be described below.

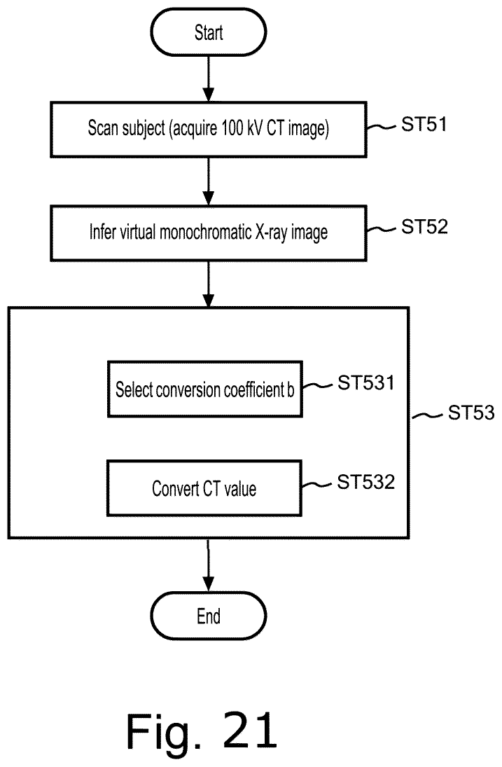

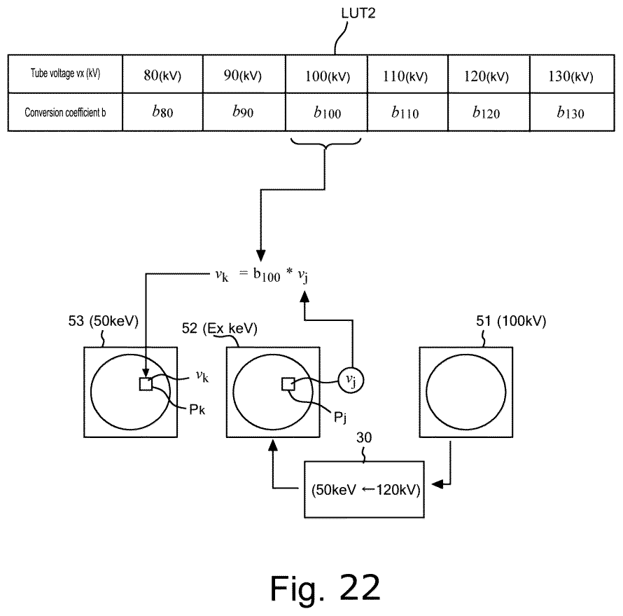

FIG. 21 is a flowchart for acquiring a 50 keV virtual monochromatic X-ray image, and FIG. 22 is an explanatory diagram of the flow of FIG. 21. In step ST51, a subject is scanned under a scanning condition in which a tube voltage of 100 kV is applied to the X-ray tube. The processor reconstructs a CT image of the subject based on data obtained by scanning the subject. FIG. 22 schematically depicts a CT image 51 acquired by scanning the subject. After scanning the subject, the process proceeds to step ST52.

In step ST52, the processor infers a virtual monochromatic X-ray image 52 from the CT image 51 using the trained neural network 30, as depicted in FIG. 22. Note that in this example, the tube voltage used to acquire the CT image 51 is 100 kV. On the other hand, the trained neural network 30 has been trained to infer a 50 keV virtual monochromatic X-ray image from a 120 kV CT image. Therefore, when the virtual monochromatic X-ray image 52 is inferred from the 100 kV CT image 51 using the trained neural network 30, as described above, the inferred virtual monochromatic X-ray image 52 is not a 50 keV virtual monochromatic X-ray image, but is a virtual monochromatic X-ray image 52 of another energy Ex offset from 50 keV by a certain energy ΔE. Therefore, a 50 keV virtual monochromatic X-ray image cannot be obtained by simply inferring the virtual monochromatic X-ray image 52 from the CT image 51 using the trained neural network 30. Therefore, in this example, the CT value of the virtual monochromatic X-ray image 52 inferred by the trained neural network 30 is converted to the CT value of a 50 keV virtual monochromatic X-ray image. In order to execute this conversion, the process proceeds to step ST53.

In step ST53, the CT value vx of the virtual monochromatic X-ray image of energy Ex is converted into a CT value v₅₀ of 50 keV using the lookup table LUT2. Step ST53 will be described below. Note that step ST53 includes steps ST531 to ST532, and thus each step will be described in order. In step ST531, the processor selects the conversion coefficient b corresponding to 100 kV from among the conversion coefficients bao to b₁₃₀ in the lookup table LUT2, as depicted in FIG. 22. 100 kV corresponds to a conversion coefficient b=b₁₀₀, and thus the processor selects b=b₁₀₀ as the conversion coefficient b corresponding to 100 kV. After selecting b=b₁₀₀, the process proceeds to step ST532. In step ST532, the processor converts the CT value vx of the virtual monochromatic X-ray image with energy Ex into the CT value v₅₀ of 50 keV based on the selected conversion coefficient b₁₀₀. Specifically, the CT value is converted as follows.

As depicted in FIG. 22, the processor identifies the pixel Pⱼ of interest from within the virtual monochromatic X-ray image 52 of energy Ex (keV). Furthermore, CT value vⱼ of a pixel Pⱼ of interest is read, and the conversion coefficient b₁₀₀ is multiplied by vⱼ. Therefore, a CT value vₖ of a pixel Pk of a 50 keV virtual monochromatic X-ray image 53 can be calculated.

In the description above, an example has been described in which the CT value vⱼ of the pixel Pⱼ of the virtual monochromatic X-ray image 52 with energy Ex (keV) is converted, but another pixel can also be converted by a similar method. Therefore, the 50 keV virtual monochromatic X-ray image 53 can be obtained. Thus, the flow ends.

In this example, after obtaining the CT image 51 obtained by a tube voltage of 100 kV, the virtual monochromatic X-ray image 52 is inferred from the CT image 51 using the trained neural network 30. However, the trained neural network 30 has been trained to infer a 50 keV virtual monochromatic X-ray image from a 120 kV CT image. Therefore, when the CT image 51 obtained with a tube voltage of 100 kV is input to the trained neural network 30, a virtual monochromatic X-ray image 52 offset from 50 keV by a constant energy is output. Therefore, in this example, in order to obtain the 50 keV virtual monochromatic X-ray image 53, the CT value of the virtual monochromatic X-ray image 52 output from the trained neural network 30 is converted into the CT value of a 50 keV virtual monochromatic X-ray image using the conversion coefficient b. Therefore, in this example, the 50 keV virtual monochromatic X-ray image 53 can be generated without preparing a new trained neural network for 100 kV in addition to the trained neural network 30 for 120 kV. Therefore, there is no need to prepare a trained neural network 30 for each tube voltage of the X-ray tube, which reduces the number of man-hours for development and significantly reduces development costs.

Furthermore, after generating the 50 keV virtual monochromatic X-ray image 53 in step ST530, a virtual monochromatic X-ray image of another energy other than 50 keV can be calculated using the lookup table LUT1 (see FIG. 14) in the same manner as in previous examples.

Note that in this example, the 100 kV CT image 51 is described as an image corresponding to a 64 keV virtual monochromatic X-ray image. However, when a subject is irradiated with X-rays, the energy properties of the X-rays absorbed by the subject vary depending on the size of the subject. Therefore, depending on the size of the subject, the 100 kV CT image 51 does not necessarily correspond to a 64 keV virtual monochromatic X-ray image. For example, in general, as the size of the subject increases, the energy of the virtual monochromatic X-ray image corresponding to a 100 kV CT image shifts toward higher energy, and as the size of the subject decreases, the energy of the virtual monochromatic X-ray image corresponding to a 100 kV CT image shifts toward lower energy. Therefore, the energy (keV) of a virtual monochromatic X-ray image corresponding to a 100 kV CT image is preferably determined based on the size of the subject. An example of the size of a subject is the diameter of an imaging site. For example, the processor can calculate a water equivalent diameter Dw based on a CT image (e.g., a scout image) of a subject, and determine the diameter of an imaging site based on the calculated water equivalent diameter Dw. Therefore, the energy of the virtual monochromatic X-ray image can be determined according to the diameter of the imaging site. Specifically, by storing, in a storing device, a plurality of diameters of imaging sites (e.g., 20 cm, 25 cm, 30 cm, 35 cm, and the like) and third lookup tables corresponding to each diameter, with the third lookup tables expressing a correspondence relationship between a plurality of tube voltages and a plurality of conversion coefficients b, a third lookup table corresponding to a determined diameter of an imaging site can be selected. Therefore, a conversion coefficient according to the tube voltage can be identified based on the selected third lookup table, and the accuracy of the estimated virtual monochromatic X-ray image can be further improved.

1. A CT system comprising:
an X-ray tube; and
one or more processors that executes the following:
reconstructing a first CT image based on data obtained by scanning a subject under a scanning condition in which a first tube voltage is applied to the X-ray tube;
inferring a virtual monochromatic X-ray image of a second energy different from a first energy corresponding to the first tube voltage based on the first CT image; and
determining a CT value of the virtual monochromatic X-ray image of a third energy based on a first coefficient defining the relationship between a second CT value corresponding to the second energy and a third CT value corresponding to the third energy, with reference to a first CT value corresponding to the first energy.

2. The CT system according to claim 1, wherein
the one or more processors uses a trained neural network to infer the virtual monochromatic X-ray image of the second energy from the first CT image.

3. The CT system according to claim 1, wherein
the one or more processors uses a first lookup table expressing a correspondence relationship between a plurality of first coefficients and a plurality of energy levels of the virtual monochromatic X-ray image to determine the CT value of the virtual monochromatic X-ray image of the third energy.

4. The CT system according to claim 3, wherein the one or more processors executes the following:
selecting a first coefficient corresponding to the first energy from the plurality of first coefficients of the first lookup table; and
generating a virtual monochromatic X-ray image of the third energy based on the selected first coefficient, the first CT image, and the virtual monochromatic X-ray image of the second energy.

5. The CT system according to claim 3, wherein generating the virtual monochromatic X-ray image of the third energy includes:
generating a difference image between the first CT image and the virtual monochromatic X-ray image of the second energy;
multiplying the CT value of the difference image by a first coefficient selected from the plurality of first coefficients to generate a multiplication image; and
adding the multiplication image to the first CT image to generate a virtual monochromatic X-ray image of the third energy.

6. The CT system according to claim 3, wherein the one or more processors executes the following:
selecting a first coefficient value corresponding to the first energy from the plurality of first coefficients of the first lookup table; and
multiplying the CT value of the difference image by the selected first coefficient to generate a multiplication image.

7. The CT system according to claim 1, wherein the one or more processors executes the following:
reconstructing a second CT image based on data obtained by scanning a subject under a scanning condition in which a second tube voltage is applied to the X-ray tube;
inferring a virtual monochromatic X-ray image of another energy different from the second energy from the second CT image using the trained neural network; and
converting the CT value of the virtual monochromatic X-ray image of the other energy into the CT value of the virtual monochromatic X-ray image of the second energy using a second coefficient.

8. The CT system according to claim 7, wherein the one or more processors uses a second lookup table expressing a correspondence relationship between a plurality of tube voltages and a plurality of second coefficients to convert the CT value of the virtual monochromatic X-ray image of the other energy into a CT value of the virtual monochromatic X-ray image of the second energy.

9. The CT system according to claim 3, wherein the one or more processors executes the following:
selecting a second coefficient corresponding to the second tube voltage from the plurality of second coefficients of the second lookup table; and
converting the CT value of the virtual monochromatic X-ray image of the other energy into the CT value of the virtual monochromatic X-ray image of the second energy based on the selected second coefficient.

10. The CT system according to claim 7, wherein the one or more processors uses the size of a subject and the second coefficient to convert the CT value of the virtual monochromatic X-ray image of the other energy into a CT value of the virtual monochromatic X-ray image of the second energy.

11. The CT system according to claim 10, wherein the size of the subject is a diameter of an imaging site of the subject.

12. The CT system according to claim 7, wherein the one or more processors determines the diameter of the imaging site based on a scout image of the subject.

13. The CT system according to claim 12, wherein the storing device stores:
a plurality of diameters of the imaging site; and
a third lookup table corresponding to each diameter, and expressing a correspondence relationship between the plurality of tube voltages and the plurality of second coefficients,
and
the one or more processors identifies the second coefficient corresponding to the second tube voltage based on the third lookup table corresponding to a diameter of the imaging site determined based on the scout image.

14. The CT system according to claim 7, wherein
the one or more processors obtains a water equivalent diameter based on a scout image of the subject to determine a diameter of the imaging site based on the obtained water equivalent diameter.

15. One or more non-transitory computer-readable storing medium storing an instruction, the storing medium causing the one or more processors to, when the instruction is executed by the one or more processors, execute an operation including:
reconstructing a first CT image based on data obtained by scanning a subject under a scanning condition in which a first tube voltage is applied to the X-ray tube;
inferring a virtual monochromatic X-ray image of a second energy different from a first energy corresponding to the first tube voltage based on the first CT image; and
determining a CT value of the virtual monochromatic X-ray image of a third energy based on a first coefficient defining the relationship between a second CT value corresponding to the second energy and a third CT value corresponding to the third energy, with reference to a first CT value corresponding to the first energy.
